(19) United States
(12) Patent Application Publication
(10) Pub. No.: US 2008/0188445 A1
(43) Pub. Date: Aug. 7, 2008

(54) TETRACYCLINE COMPOSITIONS FOR TOPICAL ADMINISTRATION

(75) Inventors: BRENDAN MULDOON, GLENGORMLEY (IE); DAVID WOOLFSON, BELFAST (IE); STEPHEN MCCULLAGH, BELFAST (IE)

Correspondence Address: FITZPATRICK CELLA HARPER & SCINTO, 30 ROCKEFELLER PLAZA, NEW YORK, NY, 10112, US

(73) Assignee: WARNER CHILCOTT COMPANY INC., Fajardo, PR (US)

(21) Appl. No.: 12/024,794

(22) Filed: Feb. 1, 2008

Related U.S. Application Data
(60) Provisional application No. 60/887,867, filed on Feb. 2, 2007.

(51) Int. Cl. A61K 31/65; A61P 17/10

(52) U.S. Cl. 514/153; 514/152

(57) ABSTRACT

Multi-part pharmaceutical formulations containing tetracycline for topical administration, as well as methods of making and administering the same, are disclosed.

This application claims the benefit of U.S. Provisional Application No. 60/887,867, filed Feb. 2, 2007.

### BACKGROUND OF THE INVENTION

1. Field of the Invention

This invention relates to multi-part tetracycline formulations for topical administration, as well as to methods of making and administering the same.

2. Related Background Art

Topical antibiotics are a widely accepted, effective and well tolerated treatment for dermatological conditions, including inflammatory acne vulgaris. Topical antibiotics for the treatment of such dermatological conditions offer the advantage of a decreased total absorption of the drug and an accompanying decrease in toxicity, when compared with systemic antibiotics. In addition, topical antibiotics offer the added benefit of applying the antibiotic directly to the targeted lesions.

Topical antibiotics commonly prescribed in the United States are clindamycin and erythromycin. Tetracycline antibiotics are also used in the treatment of dermatological conditions, but topical formulations of tetracycline antibiotics are limited. Meclocycline (an oxytetracycline derivative) has been formulated as a 1% cream (Meclan® or Meclosorb®). Tetracycline hydrochloride (0.22% w/w) is marketed in the United Kingdom under the brand name Topicycline®; this comprises an aqueous ethanol solution of tetracycline hydrochloride in equilibrium with its degradation product, 4-epitetracycline hydrochloride. The product must be reconstituted by mixing tetracycline hydrochloride powder with an aqueous ethanol solution prior to dispensing, whereupon it is only stable for 8 weeks.

Tetracyclines have limited stability in aqueous solutions (A. Kubis et al., “Investigation of stability of tetracycline hydrochloride in methylcellulose gel”, Pharmazie 42:519-520 (1987)). Tetracycline antibiotics are known to be oxidatively unstable and often change from yellow to brown over time (Y. Liang et al., “Stability studies of tetracycline in methanol solution”, J. Chromatography 827:45-55 (1998)). Despite this, efforts have been made in the prior art to formulate tetracycline compositions for topical administration. These efforts have been hindered, however, by the instability of the tetracycline compositions in the presence of water and other protic liquids. As used herein, “protic liquid” refers to any liquid that carries a hydrogen attached to an oxygen (such as in a hydroxyl group), to a nitrogen (such as in an amine group) and further including any molecular liquid which contains dissociable H⁺. In tetracycline formulations in the presence of water and other protic liquids, the tetracyclines typically form various degradation products such as, but not limited to, epitetracycline, anhydrotetracycline, epianhydrotetracycline, which degradation products have negligible antibacterial activity. This leads to a limited, commercially undesirable shelf life for such tetracycline products in aqueous media.

To overcome the stability problem, the tetracycline antibiotics have been incorporated into various nonaqueous vehicles. Solutions of tetracycline antibiotics in alcohol-based solvents are disclosed in, for example, U.S. Pat. Nos. 3,219,529, 3,389,174 and 4,376,118. However, the use of such alcohol-based solvents has not been pharmaceutically acceptable due to the instability of tetracyclines in the presence of water and other protic liquids.

The tetracycline antibiotics have also been formulated in nonaqueous ointment bases, which are less desirable in the treatment of acne due to their greasy consistency. This greasiness can, in turn, be associated with poor patient compliance.

U.S. Pat. No. 3,952,099 discloses a two component anti-acne composition. One component comprises a fluid ointment base containing a penetration enhancer, and the second component comprises a separately packaged, dry, water-proof packet containing an anti-acne agent. The anti-acne powder and the ointment base are mixed by the user shortly before use.

European Patent Publication No. 0 052 404 describes a topical composition for treating acne comprising two components. The first component is a dry tetracycline-containing powder mixture in a waterproof foil packet, and the second component contains a sulfoxide and a sugar ester in water/ethanol, the first component being added to the second component before use.

U.S. Pat. Nos. 4,497,794 and 4,692,329 describe two containers, the first container comprising an aqueous-based solution and the second container comprising antibiotic powder. Certain aqueous gel compositions comprising benzoyl peroxide, erythromycin and, optionally, dioctyl sodium sulfosuccinate are disclosed therein.

All of these patents concern themselves with separating a dry antibiotic powder from the balance of the components during storage. Such dry powder systems are inconvenient for the user, since it is difficult to ensure both no loss of powder and homogeneity of mixing during reconstitution. It is, therefore, difficult to ensure that the desired dose of active ingredient is being used. None of these patents discloses or suggests how a tetracycline antibiotic, which is not provided as a dry powder, might be stabilized in one formulation and then solubilized by subsequent mixing with another component.

U.S. Pat. No. 5,446,028 relates to a topical composition comprising a peroxide and a lincomycin antibiotic, as well as an aqueous gel composition comprising a peroxide, a lincomycin and a surfactant. Topical tetracycline administration is also described. The examples provide an antibiotic solution in a first container and the balance in a second container. However, certain examples disclose the presence of water and/or ethanol with the antibiotic in the first container. Such formulations would not stabilize a tetracycline, due to the presence of water and other protic liquids.

U.S. Pat. Nos. 5,562,642, 5,417,674 and 5,254,109 relate to a dispensing and applicator system comprising first and second moisture cover sheets and first and second applicator pads, impregnated with first and second dermatological agents, respectively. The first agent can be a peroxide and the second agent can be tetracycline. However, the tetracycline is dissolved in an aqueous, alcoholic or aqueous/alcoholic formulation. A tetracycline would not be stabilized in such a formulation, due to the presence of water and other protic liquids.

WO 99/02133 and corresponding U.S. Pat. No. 6,448,233 concern topical delivery of benzoyl peroxide and a second active agent via a multi-compartment dispensing system. The dispensing system for benzoyl peroxide and an antimicrobial agent, such as a macrolide or an aminoglycoside, may comprise a first container in which the benzoyl peroxide is suspended in an aqueous medium and a second container in which the macrolide or aminoglycoside antimicrobial agent is present in a solvent such as a glycol/alcoholic gel. However, since protic liquids are employed in the second container, a tetracycline would not be expected to be stable in such a formulation.

WO 01/91726 concerns a package comprising first and second components, one of the components being an oxidizing agent and the other being an antibiotic, the components being separated from one another in the package. The antibiotic is either dissolved in alcohol or in water. A tetracycline would not be stabilized in such a formulation, due to the presence of water and other protic liquids.

U.S. Pat. No. 6,462,025 concerns an apparatus having first and second chambers, the first chamber containing a first composition which is substantially anhydrous and includes a protic liquid, an antibiotic and a thickening agent selected from acrylic acid polymers, polyacrylamides and combinations and a second chamber containing a second composition comprising benzoyl peroxide. In related U.S. Pat. No. 7,060,732, the first chamber contains an active agent effective in treating acne (not a retinoid), and the second chamber contains a retinoid. Since protic liquids such as propylene glycol (PG) and polyethylene glycol (PEG) are employed in the first composition, a tetracycline would not be stable therein.

None of the above-mentioned patents or patent applications discloses a multi-part system, in which the stability of a tetracycline antibiotic in a first component is addressed. Thus, there is a need for a topical tetracycline composition, which is stable and convenient to use, which provides good delivery of the tetracycline to the skin surface, and which is cosmetically acceptable. Accordingly, tetracycline compositions for topical administration, that do not suffer from the deficiencies of conventional topical compositions, are desirable.

### SUMMARY OF THE INVENTION

The present invention is directed to a multi-part tetracycline formulation comprising (a) a first component containing at least one tetracycline or a pharmaceutically acceptable salt or hydrate thereof substantially stabilized in a first base; and (b) a second component containing at least a second base, wherein, upon mixture of the first component and the second component, the at least one tetracycline is rendered suitable for topical administration. In a preferred embodiment of the invention, topical administration is external administration to the skin. In another preferred embodiment, the at least one tetracycline is substantially solubilized upon mixture of the first and second components. In further preferred embodiments of the multi-part tetracycline formulation of the present invention, the first component is substantially free of any protic liquid (including water); in still other embodiments, the second component contains water and/or other protic liquids. In certain embodiments, the multi-part tetracycline formulation optionally comprises at least one penetration enhancer, at least one preservative, at least one surfactant, at least one pharmaceutically acceptable excipient, at least one mucoadhesive agent, at least one chelating agent, at least one antioxidant and/or at least one additional pharmaceutically active agent.

Preferred embodiments of the invention include those in which the at least one tetracycline comprises a 1,4,4a,5,5a,6,11,12a-octahydro naphthacene-2-carboxamide structure having two different substituents at one or more of positions 1, 4, 5, 6 and 11; hydrogen being considered a substituent. Preferably the at least one tetracycline has two different substituents at position 4 and, more preferably, the at least one tetracycline has two different substituents at each of positions 4 and 6. Tetracyclines suitable for use in the present invention are those which are unstable in water and other protic liquids. Such tetracyclines include [4S-(4α,4aα,5aα,12aα)]-4-(dimethylamino)-1,4,4a,5,5a,6,11,12a-octahydro-3,10,12,12a-tetrahydroxy-1,11-dioxo-2-naphthacenecarboxamides having two different substituents at one or more of positions 4, 5, and 6. Preferably the at least one tetracycline has two different substituents at position 4 and, more preferably, the at least one tetracycline has two different substituents at each of positions 4 and 6. More preferred embodiments of the invention include those in which the at least one tetracycline has the structural formula:

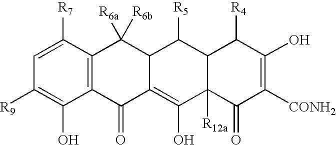

wherein R₄ is selected from the group consisting of a mono(lower alkyl)amino and a di(lower alkyl)amino;

R₉ is selected from the group consisting of hydrogen, a mono(lower alkyl)amino, a di(lower alkyl)amino and 2-(tert-butylamino)acetamido;

R₅ and R₁₂ₐ are independently selected from the group consisting of hydrogen and hydroxyl;

R₆ₐ and R_{6b} are independently selected from the group consisting of hydrogen, lower alkyl and hydroxyl, or can together form ═CH₂;

R₇ is selected from the group consisting of hydrogen, a halogen such as chloride, a mono(lower alkyl)amino and a di(lower alkyl)amino;

or a pharmaceutically acceptable salt or hydrate thereof. In a more preferred embodiment, the at least one tetracycline is selected from the group consisting of doxycycline and minocycline and their pharmaceutically acceptable salts or hydrates. In a still more preferred embodiment, the at least one tetracycline is minocycline and a pharmaceutically acceptable salt or hydrate thereof.

In an optional embodiment of the invention, the first base comprises at least one hydrophobic, non-hygroscopic silicone liquid; and at least one hydrophobic, non-hygroscopic silicone thickening agent, and wherein the first base is substantially free of protic liquids. In a further optional embodiment of the invention, the first base consists essentially of at least one hydrophobic, non-hygroscopic silicone liquid; at least one hydrophobic, non-hygroscopic silicone thickening agent and at least one penetration enhancer, and wherein the first base is substantially free of protic liquids. Optionally, the at least one penetration enhancer is present.

The present invention is also directed to a method of making a multi-part tetracycline formulation comprising the steps of: (a) preparing a first component containing at least one tetracycline or a pharmaceutically acceptable salt or hydrate thereof substantially stabilized in a first base; and (b) preparing a second component containing at least a second base. The method of the invention may further comprise the step of: (c) mixing the first component and the second component to render the at least one tetracycline suitable for topical administration. Mixing together of the first and second components can be accomplished by any suitable method using any suitable manual or automated means.

The present invention is further directed to multi-part tetracycline formulations made according to the methods of the invention.

The present invention is still further directed to a method of treating a dermatological condition comprising the step of administering a multi-part tetracycline formulation to an accessible body surface of a human or an animal in need of such treatment, wherein the multi-part tetracycline formulation comprises (a) a first component containing at least one tetracycline or a pharmaceutically acceptable salt or hydrate thereof substantially stabilized in a first base and (b) a second component containing at least a second base, and wherein, upon mixture of the first component and the second component, the at least one tetracycline is rendered suitable for topical administration. In certain embodiments, the first component and the second component are administered simultaneously; in other embodiments, the first component and the second component are administered sequentially in either order. In still other embodiments, the first component and the second component are mixed together before administering to the accessible body surface.

### BRIEF DESCRIPTION OF THE FIGURES

FIG. 1 illustrates a conventional side-by-side permeation apparatus set-up (exploded view).

FIG. 2 illustrates a conventional Franz cell apparatus.

### DETAILED DESCRIPTION OF THE INVENTION

The present invention is directed to a multi-part tetracycline formulation comprising: (a) a first component containing at least one tetracycline or a pharmaceutically acceptable salt or hydrate thereof substantially stabilized in a first base; and (b) a second component containing at least a second base, wherein, upon mixture of the first component and the second component, the at least one tetracycline is rendered suitable for topical administration. In a preferred embodiment of the present invention, the formulation is suitable for external administration to the skin.

The first component of the multi-part tetracycline formulation contains at least one tetracycline or a pharmaceutically acceptable salt or hydrate thereof substantially stabilized in a first base. As used herein, at least one tetracycline that is “substantially stabilized” in a first component refers to a first component in which preferably more than about 85%, and more preferably more than about 90%, of the at least one tetracycline or its pharmaceutically acceptable salt or hydrate remains after storage at 25° C. and 60% relative humidity (RH) for preferably about 3 months, more preferably about 6 months, and still more preferably about 12 months. “Substantially stabilized” can also refer to a formulation in which preferably more than about 85%, and more preferably more than about 90%, of the at least one tetracycline or its pharmaceutically acceptable salt or hydrate retains its antibiotic activity after storage at 25° C. and 60% relative humidity for preferably about 3 months, more preferably about 6 months, and still more preferably about 12 months. In a preferred embodiment of the invention, the at least one tetracycline is substantially suspended in the first base. As used herein, “substantially suspended” means preferably at least about 50%, more preferably at least about 75%, still more preferably at least about 85%, and most preferably at least about 95%, of the at least one tetracycline or its pharmaceutically acceptable salt or hydrate is suspended in the first base at about 32° C.

In these preferred embodiments of the invention, in which the at least one tetracycline is substantially suspended in the first base, the first base can be substantially free of any surfactant. As used herein, “substantially free” refers to the presence of preferably less than about 2%, more preferably less than about 1.5%, still more preferably less than about 1%, still further more preferably less than about 0.2%, and most preferably less than about 0.02%, w/w surfactants. Without being bound by theory, it is thought that the at least one tetracycline can be substantially stabilized by being substantially suspended and, therefore, physically separated from those agents that cause a reduction in antibiotic activity. For this reason, in the preferred embodiments of the invention, the use of surfactants in the first base is undesirable, the aim, instead, being to maintain the at least one tetracycline substantially in suspension in the first base.

“Tetracycline” refers to a number of antibiotics derived from a system of four linearly annelated six-membered rings (1,4,4a,5,5a,6,11,12a-octahydronaphthacene) with a characteristic arrangement of double bonds. Certain known tetracyclines comprise a 1,4,4a,5,5a,6,11,12a-octahydro naphthacene-2-carboxamide structure having two different substituents at one or more of positions 1, 4, 5, 6 and 11; hydrogen is considered a substituent. Preferably the at least one tetracycline has two different substituents at position 4 and, more preferably, the at least one tetracycline has two different substituents at each of positions 4 and 6. Tetracyclines suitable for use in the present invention are those which are unstable in water and other protic liquids. Such tetracyclines include [4S-(4α,4aα,5aα,12aα)]-4-(dimethylamino)-1,4,4a,5,5a,6,11,12a-octahydro-3,10,12,12a-tetrahydroxy-1,11-dioxo-2-naphthacenecarboxamides having two different substituents at one or more of positions 4, 5, and 6 Preferably the at least one tetracycline has two different substituents at position 4 and, more preferably, the at least one tetracycline has two different substituents at each of positions 4 and 6. More preferably such tetracyclines include, without limitation, those having the following structural formula:

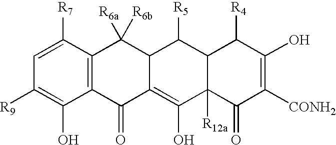

in which R₄ is selected from the group consisting of a mono(lower alkyl)amino and a di(lower alkyl)amino;

R₉ is selected from the group consisting of hydrogen, a mono(lower alkyl)amino, a di(lower alkyl)amino and 2-(tert-butylamino)acetamido;

R₅ and R₁₂ₐ are independently selected from the group consisting of hydrogen and hydroxyl;

R₆ₐ and R_{6b} are independently selected from the group consisting of hydrogen, lower alkyl and hydroxyl, or can together form ═CH₂;

R₇ is selected from the group consisting of hydrogen, a halogen such as chloride, a mono(lower alkyl)amino and a di(lower alkyl)amino; or a pharmaceutically acceptable salt or hydrate thereof.

The presence of a mono- or di-(lower alkyl)amino substituent at R₄ is believed to render a tetracycline unstable to water and other protic liquids—the 4-epimer degradation product has negligible antibacterial activity. The 6-epimer can form when the R₆ₐ and R_{6b} substituents are different. Tetracyclines suitable for use in this invention also include pharmaceutically acceptable salts and hydrates of suitable tetracyclines, in particular, but not limited to non-toxic acid addition salts such as hydrochloric, sulfonic and trichloroacetic acid salts. Tetracyclines suitable for use in the present invention also include prodrugs and derivatives thereof, provided they share the naphthacene core structure and include at least one substituent that is unstable to water and other protic liquids.

Exemplary tetracyclines represented by the above structural formula include, without limitation, tetracycline; 7-methylamino-6-deoxy-6-demethyltetracycline; 7-ethylamino-6-deoxy-6-demethyltetracycline; 7-isopropylamino-6-deoxy-6-demethyltetracycline; 9-methylamino-6-deoxy-6-demethyltetracycline; 9-ethylamino-6-deoxy-6-demethyltetracycline; 9-isopropylamino-6-deoxy-6-demethyltetracycline; 7,9-di(ethylamino)-6-deoxy-6-demethyltetracycline; 7-dimethylamino-6-deoxy-6-demethyltetracycline (minocycline); 9-dimethylamino-6-deoxy-6-demethyltetracycline; 7-methylamino-6-deoxytetracycline; 9-ethylamino-6-deoxytetracycline; 7,9-di(methylamino)-6-deoxytetracycline; 7-diethylamino-6-deoxytetracycline; 9-diethylamino-6-deoxytetracycline; 7,9-di(methylethylamino)-6-deoxytetracycline; 7-methylamino-9-ethylamino-6-deoxytetracycline; 9-methylamino-5-hydroxy-6-deoxytetracycline; 6-deoxy-5-hydroxytetracycline (doxycycline); oxytetracycline; 7-chlorotetracycline; 7-chloro-6-demethyltetracycline; 6-methyleneoxytetracycline; tigecycline and the pharmaceutically acceptable salts and hydrates of the foregoing.

More preferred tetracyclines include, without limitation, tetracycline; 7-dimethylamino-6-deoxy-6-demethyltetracycline; 7-methylamino-6-deoxy-6-demethyl-tetracycline; 9-methylamino-6-deoxy-6-demethyltetracycline; 7-ethylamino-6-deoxy-6-demethyltetracycline; 7-isopropylamino-6-deoxy-6-demethyltetracycline; 6-deoxy-5-hydroxytetracycline; oxytetracycline; 7-chlorotetracycline; 7-chloro-6-demethyltetracycline; 6-methyleneoxytetracycline; tigecycline and the pharmaceutically acceptable salts and hydrates of the foregoing.

Specific examples of the most preferred tetracyclines include, without limitation, tetracycline, minocycline, doxycycline, oxytetracycline, chlortetracycline, demeclocycline, methacycline, tigecycline, and the pharmaceutically acceptable salts or hydrates of the foregoing. Special mention is made of minocycline and doxycycline, and their pharmaceutically acceptable salts or hydrates. Minocycline and its salts and hydrates are especially preferred for use in the present invention. Minocycline is a potent semi-synthetic tetracycline with activity against a wide range of gram-positive and gram-negative organisms. It has been shown to be particularly effective as adjunctive therapy in the treatment of severe acne.

According to the present invention, the at least one tetracycline is preferably employed in an amount ranging from about 0.00001% to about 10%, more preferably in an amount ranging from about 0.0025% to about 6%, and most preferably in an amount ranging from about 0.01% to about 3%, by weight of the multi-part tetracycline formulation. Since the first and second components are typically mixed in equal parts, in other words, the at least one tetracyline is preferably employed in the first component in an amount ranging from about 0.00002% to about 20%, more preferably in an amount ranging from about 0.005% to about 12%, and most preferably in an amount ranging from about 0.02% to about 6%, by weight of the first component.

Bases suitable for use as the first base in the first component may be a hydrophobic, non-hygroscopic liquid; a semi-solid hydrophobic, non-hygroscopic vehicle; or a combination thereof. As used herein, “non-hygroscopic” refers to a material which does not readily take up water. As used herein, “hydrophobic” refers to being non-polar and thus having no affinity for water. Suitable hydrophobic and non-hygroscopic first bases (and their individual constituents) can have a contact angle of greater than about 90 degrees. First bases suitable for use comprise liquid vehicles and semi-solid vehicles or combinations thereof. In a preferred embodiment, the first base comprises at least one hydrophobic, non-hygroscopic liquid and at least one hydrophobic, non-hygroscopic semi-solid vehicle, such as a silicone thickening agent.

Suitable hydrophobic, non-hygroscopic liquid vehicles for use in the first base include, without limitation, mineral oils, silicone liquids, non-protic liquids such as, without limitation, decylmethyl sulfoxide and dialkyl isosorbides such as dimethyl isosorbide, and combinations thereof. As used herein, “non-protic liquids” refer to liquids that share ion dissolving power with protic liquids but which lack the dissociable H⁺, otherwise known as the acidic hydrogen of a polar liquid. In contrast, protic liquids do have such a dissociable H⁺, for example, a hydrogen attached to an oxygen (such as in a hydroxyl group) or to a nitrogen (such as in an amine group).

Suitable silicone liquids include, without limitation, linear and cyclic siloxane polymers and copolymers, for example, alkyl, haloalkyl and aryl, linear and cyclic, siloxane polymers and copolymers. For example, suitable silicone liquids include, without limitation, linear and cyclic alkyl and aryl siloxanes such as linear polydimethylsiloxane (commonly known as silicone oil), cyclopolydimethylsiloxanes (cyclomethicones) including, but not limited to, decamethylcyclopentasiloxane, further including, without limitation, low molecular weight linear and cyclic volatile methyl siloxanes; low molecular weight linear and cyclic volatile and non-volatile alkyl and aryl siloxanes; and low molecular weight linear and cyclic functionalised siloxanes. Also included within the scope of functionalised silicone liquids are halosilicone liquids, including fluorosilicone liquids, further including, without limitation, trifluoropropylmethyl siloxane. Also included within the scope of silicone liquids are copolymers thereof, including, without limitation, dimethylsiloxane and trifluoropropylmethylsiloxane copolymers supplied by, for example, Nusil. Further included within the scope of functionalised silicone liquids are hydride- and vinyl-functionalised silicone liquids, including, without limitation, hydride- and vinyl-functionalised linear and cyclic alkyl, haloalkyl and aryl siloxane polymers and copolymers.

Low molecular weight linear and cyclic volatile methyl siloxanes (VMS) are considered as suitable silicone liquids. VMS compounds correspond to the average unit formula (CH₃)ₐ SiO₍₄₋ₐ₎/2 in which a has an average value of two to three. The compounds contain siloxane units joined by ≡Si—O—Si≡ bonds. Representative units are monofunctional “M” units (CH₃)₃ SiO₁/2 and difunctional “D” units (CH₃)₂ SiO₂/2. The presence of trifunctional “T” units CH₃ SiO₃/2 results in the formation of branched linear or cyclic volatile methyl siloxanes. The presence of tetrafunctional “Q” units SiO₄/2 results in the formation of branched linear or cyclic volatile methyl siloxanes.

Linear VMS have the formula (CH₃)₃ SiO{(CH₃)₂ SiO}_{y} Si(CH₃)₃. The value of y is 0-5. Cyclic VMS have the formula {(CH₃)₂ SiO}_{z}. The value of z is 3-6. Preferably, these volatile methyl siloxanes have boiling points less than about 250° C. and viscosities of about 0.65-5.0 centistokes (mm²/s). Representative linear volatile methyl siloxanes are hexamethyldisiloxane (MM) with a boiling point of 100° C., viscosity of 0.65 mm²/s, and formula Me₃ SiOSiMe₃; octamethyltrisiloxane (MDM) with a boiling point of 152° C., viscosity of 1.04 mm²/s, and formula Me₃ SiOMe₂ SiOSiMe₃ decamethyltetrasiloxane (MD₂ M) with a boiling point of 194° C., viscosity of 1.53 mm²/s, and formula Me₃ SiO(Me₂ SiO)₂ SiMe₃; dodecamethylpentasiloxane (MD₃ M) with a boiling point of 229° C., viscosity of 2.06 mm²/s, and formula Me₃ SiO(Me₂ SiO)₃ SiMe₃; tetradecamethylhexasiloxane (MD₄ M) with a boiling point of 245° C., viscosity of 2.63 mm²/s, and formula Me₃ SiO(Me₂ SiO)₄ SiMe₃; and hexadecamethylheptasiloxane (MD₅ M) with a boiling point of 270° C., viscosity of 3.24 mm²/s, and formula Me₃ SiO(Me₂ SiO)₅ SiMe₃. Representative cyclic volatile methyl siloxanes are hexamethylcyclotrisiloxane (D₃) a solid with a boiling point of 134° C. and formula {(Me₂)_{SiO}}₃; octamethylcyclotetrasiloxane (D₄) with a boiling point of 176° C., viscosity of 2.3 mm²/s, and formula {(Me₂)_{SiO}}₄; decamethylcyclopentasiloxane (D₅) with a boiling point of 210° C., viscosity of 3.87 mm²/s, and formula {(Me₂)_{SiO}}₅; and dodecamethylcyclohexasiloxane (D₆) with a boiling point of 245° C., viscosity of 6.62 mm²/s, and formula {(Me₂)_{SiO}}₆. Representative branched volatile methyl siloxanes and are heptamethyl-3-{(trimethylsilyl)oxy}trisiloxane (M₃ T) with a boiling point of 192° C., viscosity of 1.57 mm²/s, and formula C₁₀H₃₀O₃Si₄ hexamethyl-3,3,bis{(trimethylsilyl)oxy}trisiloxane (M₄ Q) with a boiling point of 222° C., viscosity of 2.86 mm²/s, and formula C₁₂H₃₆O₄Si₅; and pentamethyl {(trimethylsilyl)oxy}cyclotrisiloxane (MD₃) with the formula C₈H₂₄O₄Si₄. Low molecular weight linear and cyclic volatile and non-volatile alkyl and aryl siloxane can also be used. Representative linear polysiloxanes are compounds of the formula R₃ SiO(R₂ SiO)_{y} SiR₃, and representative cyclic polysiloxanes are compounds of the formula (R₂ SiO)_{z}. R is an alkyl group of 1-6 carbon atoms, or an aryl group such as phenyl. The value of y is 0-80, optionally 0-20. The value of z is 0-9, optionally 4-6. These polysiloxanes have viscosities generally in the range of about 1-100 centistokes (mm²/s).

Suitable semi-solid vehicles for use in the first component include, without limitation, silicone-based elastomers, hydrocarbon waxes, colloidal silicon dioxide, magnesium aluminum silicate, hydrocarbon ointment bases (such as Plastibase) and combinations thereof. Particularly preferred bases for the first component include the chemically crosslinked ST-Elastomer 9041 (a silicone elastomer in dodecamethyl pentasiloxane) and ST-Elastomer 10 (also known as Dow Corning 9040 Silicone Elastomer Blend; mixture of high molecular weight silicone elastomer (12%) in decamethylcyclopentasiloxane (D5)) and Plastibase® (Squibb; also known as Jelene®; 5% w/w low molecular weight polyethylene/95% w/w mineral oil mix). ST-Elastomer 10 is most preferred.

Suitable hydrophobic, non-hygroscopic semi-solid vehicles for use in the first component can comprise silicone thickening agents, and combinations thereof. Silicone thickening agents partly or wholly comprise one or more polysiloxane-derived components. A polysiloxane-derived component is defined as any constituent comprising the general chemical motif —[Si(R¹)(R²)—O]ₙ—, in which n defines the number of repeat units (chemical motifs) in the polysiloxane and may take values in the range from about 5 to about 1,000,000; in which part or all of the backbone of the polysiloxane-derived component comprises alternating silicon (S) and oxygen (O) atoms; and in which R¹ and R² groups, which may be the same or different, are selected from a wide range of chemical ligands known in the art. Examples include, but are not limited to, alkyl, vinyl, hydrogen, aryl and fluoride ligands. Preferably, the R¹ and R² groups, which are the same or different, are alkyl groups such that the silicone thickening agent is nominally derived from polydialkylsiloxane, and most preferably the R¹ and R² groups are each methyl ligands, such that the silicone thickening agent is nominally derived from polydimethylsiloxane. Optionally, the silicone thickening agent(s) or combinations thereof, may be chemically crosslinked according to methods known by those skilled in the art. Alternatively, the silicone thickening agent may be an amino-functional silicone. Such silicones are cationic silicones with an enhanced ability to bind to keratinaceous substrates. Further alternatively, the silicone thickening agent may be an anionic silicone.

Preferred semi-solid, hydrophobic, non-hygroscopic vehicles for use in the first component include silicone elastomers, and combinations thereof, wherein the at least one polysiloxane-derived component is physically or chemically crosslinked to form a three-dimensional polymeric network. If the multi-part formulation is in the form of a gel, paste or ointment, the at least one silicone elastomer, or more generally the at least one semi-solid, hydrophobic, non-hygroscopic vehicle, comprises at least 5% w/w, preferably greater than 7.5% w/w, and more preferably between 7.5 and 15% w/w, of the overall formulation. If the multi-part formulation is in the form of a lotion, the at least one silicone elastomer, or more generally the at least one semi-solid, hydrophobic, non-hygroscopic vehicle, comprises at least 0.5% w/w, optionally at least 1% w/w, and further optionally at least 2% w/w of the overall formulation.

Silicone elastomers can be prepared by a crosslinking reaction between (A)≡Si—H containing polysiloxanes and (B) an alpha, omega-diene in the presence of a platinum catalyst and (C) a low molecular weight linear or cyclic polysiloxane, as described in U.S. Pat. No. 5,654,362. The elastomers can be swollen with a low molecular weight polysiloxane under a shear force.

Suitable silicone-based vehicles can be prepared as described in U.S. Pat. No. 5,654,362 and International Patent Publication No. WO 2006/138035, the disclosures of which are hereby incorporated by reference in their entirety. More specifically, silicone oils or other solvents can be thickened to a gel-like consistency by reacting (A) a≡Si—H containing polysiloxane of formula R₃ SiO(R′₂ SiO)ₐ (R″HSiO)_{b} SiR₃ and optionally a —Si—H containing polysiloxane of formula HR₂ SiO(R′₂ SiO)_{c} SiR₂ H or formula HR₂ SiO(R′₂ SiO)ₐ (R″HSiO)_{b} SiR₂ H where R, R′, and R″ are alkyl groups with 1-6 carbon atoms; a is 0-250; b is 1-250; and c is 0-250; with (B) an alpha, omega-diene of formula CH₂═CH(CH₂)ₓ CH═CH₂ where x is 1-20. The reaction is conducted in the presence of a platinum catalyst, in the presence of (C) a low molecular weight silicone oil or other solvent. The reaction is continued until a gel is formed by crosslinking and addition of ≡Si—H across double bonds in the alpha, omega-diene.

The ≡Si—H containing polysiloxane (A) is represented by compounds of the formula R₃ SiO(R′₂ SiO)ₐ (R″HSiO)_{b} SiR₃ designated as type A¹ and compounds of the formula HR₂ SiO(R′₂ SiO)_{c} SiR₂ H or formula HR₂ SiO(R′₂ SiO)ₐ (R″HSiO)_{b} SiR₂ H designated h as type A². In these formulae, R, R′, and R″, are alkyl groups with 1-6 carbon atoms; a is 0-250; b is 1-250; and c is 0-250. The reaction can be conducted using only compounds of type A¹. If both types A¹ and A² are present, the molar ratio of compounds A²: A¹ is 0-20, preferably 0-5.

The alpha, omega-diene (B) is a compound of the formula CH₂═CH(CH₂)ₓ CH═CH₂ where x is 1-20. Some representative examples of suitable alpha, omega-dienes for use herein are 1,4-pentadiene; 1,5-hexadiene; 1,6-heptadiene; 1,7-octadiene; 1,8-nonadiene; 1,9-decadiene; 1,11-dodecadiene; 1,13-tetradecadiene; and 1,19-eicosadiene.

The addition and crosslinking reaction requires a catalyst to effect the reaction between the ≡SiH containing polysiloxane and the alpha, omega-diene. Suitable catalysts are Group VIII transition metals, i.e., the noble metals. Such noble metal catalysts are described in U.S. Pat. No. 3,923,705, incorporated herein by reference to show platinum catalysts. One platinum catalyst is Karstedt's catalyst, which is described in Karstedt's U.S. Pat. Nos. 3,715,334 and 3,814,730, incorporated herein by reference. Karstedt's catalyst is a platinum divinyl tetramethyl disiloxane complex typically containing about one weight percent of platinum in a solvent such as toluene. Another platinum catalyst is a reaction product of chloroplatinic acid and an organosilicon compound containing terminal aliphatic unsaturation and is described in U.S. Pat. No. 3,419,593, incorporated herein by reference. The noble metal catalysts are used in amounts from 0.00001-0.5 parts per 100 weight parts of the ≡SiH containing polysiloxane, preferably 0.00001-0.02 parts, most preferably 0.00001-0.002 parts.

The phrase low molecular weight silicone oil (C) includes (i) low molecular weight linear and cyclic volatile methyl siloxanes, (ii) low molecular weight linear and cyclic volatile and non-volatile alkyl and aryl siloxanes, and (iii) low molecular weight linear and cyclic functional siloxanes; these materials are described above with regard to suitable silicone liquids.

Other suitable silicone thickening agents comprise copolymers comprising a polysiloxane (including, but not limited to, a polydimethylsiloxane) and an ester, an amide or an ether, including, but not limited to, polyoxyalkylene ether.

Still further suitable silicone thickening agents comprise graft copolymers comprising a polysiloxane (including, but not limited to, a polydimethylsiloxane) and polyvinyls, polyethylene, polypropylene, polystyrene, polyacrylates and polyurethanes.

Silicone-based thickening agents seem to be associated with improved skin feel. It is postulated that silicones provide a silky skin feel, by reducing tack and improving spreading, but without greasiness. Without being bound by theory, it is expected that the improved skin feel and the decreased perception of greasiness should improve user compliance.

The base of the first component, and indeed, the entire first component, should be substantially free of protic liquids, such as water and as defined above. As used herein, “substantially free” refers to the presence of preferably less than about 10.0%, more preferably less than about 5.0%, still more preferably less than about 2.5%, and most preferably less than about 0.75% w/w protic liquids. “Substantially free” can also refer to the presence of less than about 0.75% w/w, and more preferably less than about 0.5% w/w free water. As used herein, “free water” refers to water not associated with the tetracycline or its pharmaceutically acceptable salt. Examples of protic liquids include, but are not limited to, water, alcohols such as methanol, ethanol, glycerol, polyhydric alcohols and glycols such as ethylene glycol, propylene glycol and polyethylene glycol, acids such as acetic acid and formic acid, and bases such as ammonia.

It is preferred that the second base should include water or a protic liquid. Bases suitable for use as the second base in the second component may be a liquid (protic or non-protic, aqueous or non-aqueous), a semi-solid vehicle or a combination thereof. Suitable liquids for the second base include pharmaceutically acceptable water-miscible liquids including protic liquids such as, without limitation, water, polyethylene glycol, propylene glycol, ethanol, buffering solutions and combinations thereof and non-protic liquids such as, without limitation, decylmethyl sulfoxide and dialkyl isosorbides such as dimethyl isosorbide, and combinations thereof.

Suitable semi-solid vehicles or structural components comprise hydrophilic, hygroscopic or non-hygroscopic, semi-solid vehicles, such as, without limitation, polyethylene homopolymers and copolymers, including, but not limited to, homopolymers, oxidized homopolymers, copolymers with, for example, acrylic acid copolymers and/or vinyl acetate, and mixtures thereof; the polyacrylic acids (known as the carbomers), including, but not limited to, homopolymers, oxidized homopolymers, copolymers with polyethylene copolymers and/or vinyl acetate, and mixtures thereof; and the cellulose ethers (such as hydroxyl ethyl cellulose), including, but not limited to, homopolymers, oxidized homopolymers, copolymers with acrylic acid copolymers and/or vinyl acetate, and mixtures thereof; as well as mixtures of any two or three of polyethylene homopolymers and copolymers, the polyacrylic acids (known as the carbomers) and the cellulose ethers.

The second component may optionally further include at least one pH modifier. Suitable pH modifiers include, without limitation, sodium hydroxide, triethanolamine, hydrochloric acid and combinations thereof. An amount of pH modifier used in the second component is an amount sufficient to achieve a desired pH level (as set forth below).

The first component, the second component, or both components may further include one or more optional ingredients such as mucoadhesive agents, surfactants, penetration enhancers, antioxidants, chelating agents, pharmaceutically acceptable excipients, additional pharmaceutically active agents and preservatives. When present, such optional ingredients are included in an amount, which can be readily determined by one of ordinary skill in the art. Furthermore, one of ordinary skill in the art would readily appreciate that care should be taken in selecting optional ingredients (mucoadhesive agents, surfactants, penetration enhancers, antioxidants, chelating agents, pharmaceutically acceptable excipients, additional pharmaceutically active agents and preservatives) so as not to include an ingredient in the first component which would compromise the substantial stability of the at least one tetracycline therein; in other words, certain listed optional ingredients may be more suitable for inclusion in the second component rather than in the first component, and one of ordinary skill in the art would readily distinguish between the same. It is also contemplated that optional ingredients may be contained in additional components of a multi-part tetracycline composition if so desired (or required due to stability concerns).

The multi-part tetracycline composition of the present invention may contain at least one mucoadhesive agent. As used herein, “mucoadhesive” refers to adhering to a biological substrate comprising mucosal surfaces. Suitable mucoadhesive agents include, without limitation, the copolymers of poly(methylvinylether/maleic anhydride), known commercially as Gantrez copolymers, in order to enhance the residence time of the final composition at the site of application. Mucoadhesive agents are preferably included in the second component.

A further optional ingredient in the second component is at least one surfactant. A surfactant can be classified by the presence of formally charged groups. A nonionic surfactant has no charge groups. An ionic surfactant carries a net charge, which, if negative, means the surfactant is anionic and, if positive, means the surfactant is cationic. If a surfactant contains two oppositely charged groups, it is zwitterionic. Anionic surfactants suitable for use herein include those based on phosphate, sulfate, sulfonate or carboxylate anions such as, but not limited to, sodium dodecyl sulfate (SDS), ammonium lauryl sulfate, and other alkyl sulfate salts, sodium lauryl phosphate, sodium lauryl sulfate (SLS), sodium laureth sulfate, also known as sodium lauryl ether sulfate (SLES), alkyl benzene sulfonate, soaps, sulfosuccinates such as docusate sodium. Cationic surfactants suitable for use herein include those based on quaternary ammonium cations such as, but not limited to, cetyl trimethylammonium bromide (CTAB) and other alkyltrimethylammonium salts; cetylpyridinium chloride (CPC), polyethoxylated tallow amine (POEA), benzalkonium chloride (BAC), benzethonium chloride (BZT). Zwitterionic surfactants suitable for use herein include dodecyl betaine, dodecyl dimethylamine oxide, cocamidopropyl betaine, coco ampho glycinate. Nonionic surfactants suitable for use herein include alkyl poly(ethylene oxide), alkyl polyglucosides, including octyl glucoside, decyl maltoside, fatty alcohols, cetyl alcohol, oleyl alcohol, cocamide MEA, cocamide DEA, cocamide TEA, PEGylated sorbitans esterified with fatty acids (Tweens) and poloxamers (Pluronics by BASF; block copolymers based on ethylene oxide and propylene oxide). Surfactants are preferably included in the second component. A particularly preferred surfactant for inclusion in the second component is docusate sodium.

Still another optional ingredient is at least one penetration enhancer. As used herein, “penetration enhancer” refers to an agent that alters the movement of the active ingredient across the skin, either by a direct interaction on the skin or by adjusting the physico-chemical characteristics of the active ingredient or both. Penetration enhancers suitable for use in the present invention include, without limitation, azone, dimethyl sulfoxide, oleic acid, d-limonene, or a fatty acid ester optionally formed from a fatty acid comprising from 2 to 20 carbon atoms (such as, but not limited to caproic acid, lauric acid, myristic acid, oleic acid, linoleic acid, adipic acid and lanolic acid) optionally esterified with an alcohol of 2 to 20 carbon atoms, such as an alkanol of 2 to 4 carbon atoms, menthol and the non-ionic alkoxylates (such as, but not limited to, Arlamol). Fatty acid esters are preferred penetration enhancers. A particularly preferred fatty acid ester penetration enhancer for inclusion in the first component is isopropyl myristate. Other penetration enhancers more suitable for inclusion in the second component include terpinol, cyclodextrin and alcohols such as ethanol. In a particularly preferred embodiment of the invention, the first base consists essentially of at least one hydrophobic, non-hygroscopic silicone liquid, at least one hydrophobic, non-hygroscopic silicone thickening agent and at least one penetration enhancer.

Another optional ingredient is at least one antioxidant and/or at least one chelating agent. Suitable antioxidants and chelating agents useful in the context of the present invention include, but are not limited to, ascorbic acid and its salts, citric acid and its salts, edatate and its salts and tocopherol and its derivatives.

Still another optional ingredient is at least one additional pharmaceutically acceptable excipient. Suitable excipients include, without limitation, waxes (such as white soft paraffin), poly(vinyl alcohol), hydroxyethylcellulose, hydroxypropylcellulose, hydroxypropylmethylcellulose, buffers (for example, those buffers comprising weak organic acids such as lactic acid or acetic acid), ion-pairing salts for example the alkylsulfonates, and, if the topical composition is a foam, suitable propellants such as liquefied propellants (for example, propane, isobutene, n-butane, dimethyl ether and the chlorofluorocarbons) and combinations thereof.

Another optional ingredient is at least one preservative. Suitable preservatives include, without limitation, para-hydroxybenzoate derivatives commonly known as parabens.

At least one additional pharmaceutically active agent may also be optionally included in the multi-part tetracycline formulation of the present invention. In one preferred embodiment, however, the at least one tetracycline is the only pharmaceutically active agent present. As used herein, “pharmaceutically active agent” or “agent” or “drug” or “active agent” or “active ingredient”, etc., refers to any agent capable of defending against, or treating, a disease or cosmetic state (infection control or skin disease) in the human or animal body, or a prodrug thereof. Such pharmaceutically active agents may be organic or inorganic and may be prophylactically or therapeutically active, systemically or locally. Alternatively or additionally, such pharmaceutically active agents may be cosmetically active. As used herein, “prophylactically active” refers to an agent's (or its prodrug's) effectiveness in defending against a disease state in the human or animal body, preferably the human body. As used herein, “therapeutically active” refers to an agent's (or its prodrug's) effectiveness in treating a disease state in the human or animal body, preferably the human body. As used herein, “cosmetically active” refers to an agent's (or its prodrug's) effectiveness in defending against or treating a cosmetic condition in or on the human or animal body, preferably the human body. Typically, the additional pharmaceutically active agent is selected from anti-inflammatory compounds (such as diclofenac, ibuprofen, ketoprofen), antimicrobials (such as clindamycin and erythromycin) and the like, keratolytic agents such as benzoyl peroxide, azelic acid, retinoids, calcineurin antagonists, immunomodulators, and combinations thereof. The term “retinoids” includes first generation retinoids such as retinol, tretinoin, isotretinoin and alitretinoin, second generation retinoids such as etretinate and its metabolite, acitretin, and third generation retinoids such as tazarotene and bexarotene.

The multi-part tetracycline formulation (any one component or any combination of components) may take the form of a semi-solid preparation (such as a gel, cream, paste or ointment), a pourable preparation (such as a solution or lotion), or a foam. The final form (of each individual component as well as of the mixture of the two components) requires, that the tetracycline be substantially stabilized in the first component and, upon mixture of the first component and the second component, the at least one tetracycline is rendered suitable for topical administration. It should be apparent to one of ordinary skill in the art that the final form (of each individual component as well as of the mixture of the two components) will be dependent upon the composition of the respective bases for the first and second components, i.e., presence and amounts of viscosity enhancers, solvents, etc.

As used herein, “semi-solid” is understood to refer to the rheological properties of the compositions themselves, such that the compositions will flow under an applied force but will remain in situ following application to any accessible body surface. As used herein, a “lotion” is a dermatological vehicle that is either a pourable suspension of insoluble powder in a liquid or a pourable oil-in-water emulsion. As used herein, a “gel” is a semi-solid vehicle that consists of a liquid phase that is constrained within a three-dimensional polymeric network. The polymeric network may be formed by chemical (covalent crosslinks) or physical (hydrogen bonds, Van der Waals forces) interactions between polymer chains (more correctly, between functional groups on polymer chains). Where the liquid phase of the second component is water (or water plus a water-miscible co-solvent such as ethanol), the gel is a hydrogel (hydrophilic gel). Where the liquid phase is non-aqueous, the gel is an organogel. Oleogels are lipophilic gels whose bases typically consist of liquid paraffin with polyethylene or fatty oils gelled with colloidal silica or a long-chain fatty acid soap. As used herein, an “ointment” base is a semi-solid vehicle composed of hydrophobic constituents. Ointments can take the form of hydrocarbon ointment, non-hydrocarbon ointment, an absorption base ointment or a hydrophilic ointment. Ointments related to the present invention can be formulated to provide a non-greasy, cosmetically acceptable appearance. As used herein, a “cream” is a two phase semi-solid vehicle in which a lipophilic phase and an aqueous phase that are normally immiscible together are stabilized by the use of a suitable emulsifying agent to form a single coherent vehicle in which one phase is homogeneously dispersed in the other. As used herein, a “paste” is an ointment with a high loading of insoluble solids (up to 50% by weight) that forms a structured particulate matrix. As used herein, a “foam” is a disperse system consisting of a three dimensional network of surfactant films in air. Foams have a high surface area and tend to spontaneous collapse unless stabilized.

Preferably, the first and second components are mixed in about equal weight parts, though other mixture ratios are not explicitly excluded, to form a composition for topical administration. Other suitable mixture ratios can include from about 10 parts by weight of the first component to about 90 parts by weight of the second component to about 90 parts by weight of the first component to about 10 parts by weight of the second component. Preferably, the multi-part topical composition has a pH in the range of about 4 to about 8, either in the second component, in the mixture of the first and second components or both. Preferably, the multi-part topical composition containing minocycline has a pH in the range of about 4 to about 8, more preferably of about 6 to about 7.5, still more preferably from about 6 to about 6.8, and most preferably from about 6.2 to about 6.6, either in the second component, in the mixture of the first and second components or both.

The first and second components may be separately stored in sachets or any other suitable container in a kit. Alternatively, the first and second components may be stored in separate chambers of a dual-chambered pump, the pump being constructed and arranged to release a desired weight amount of each of the first and second components from respective first and second chambers. Additionally, any other suitable container which would keep the first and second components separate prior to mixing is suitable for use with the present invention.

Upon mixture of the first and second components of the multi-part tetracycline formulation of the present invention, the tetracycline is rendered suitable for topical delivery. As used herein, “rendered suitable for topical delivery” refers to the availability of tetracycline to be absorbed by an accessible body surface and present in an amount effective to topically treat a disease condition such as acne including acne rosacea. It is desired that the tetracycline be in a molecularly dispersed form to facilitate topical delivery. In particularly preferred embodiments of the present invention, the tetracycline which is substantially stabilized in the first component is substantially solubilized by the second component upon mixture of the two components. As used herein, “substantially solubilized” refers to preferably at least about 50%, more preferably at least about 75%, still more preferably at least about 85%, and most preferably at least about 95%, of the at least one tetracycline (or its salt or hydrate) is solubilized in the topical composition, upon mixture of the first and second components at about 32° C.

The exceptional stability of the tetracycline in the first component eliminates the need for any reconstitution from a powder (i.e., there is no dry powder component in the present invention) prior to dispensing and saves the patient expense because there is no need for special storage or frequent replacement.

The present invention is further directed to a method of making a multi-part tetracycline formulation comprising the steps of: (a) preparing a first component containing at least one tetracycline or a pharmaceutically acceptable salt or hydrate thereof substantially stabilized in a first base; and (b) preparing a second component containing at least a second base. The method of the invention optionally comprises step (c) mixing the first component and the second component to render the at least one tetracycline suitable for topical administration. All of the details regarding the tetracycline, the bases, the components, etc. are the same as set forth above with regard to the first embodiment of the invention.

Preparation of Each of the First and Second Components can be accomplished by any suitable method using any suitable means, e.g., by admixture of the ingredients typically through the use of vigorous agitation such as high shear mixing. In the preparation of the second component, a pH ranging preferably from about 4 to about 8, is achieved. Mixing together of the first and second components can likewise be accomplished by any suitable method using any suitable manual or automated means. For example, simultaneous dispensation from a suitable dual-chambered container may accomplish mixture; independent dispensation from respective containers followed by mixture by any suitable means, e.g., spatula, finger, etc. will also accomplish mixture. Optional additional steps include those which result in the addition of one or more of the optional ingredients set forth above with respect to the first embodiment.

The present invention is still further directed to a multi-part tetracycline formulation made according to the methods of the second embodiment of the invention.

Another embodiment of the invention is directed to a method of treating a dermatological condition comprising the step of administering a multi-part tetracycline formulation to an accessible body surface of a human or an animal in need of such treatment, wherein the multi-part tetracycline formulation comprises (a) a first component containing at least one tetracycline or a pharmaceutically acceptable salt or hydrate thereof substantially stabilized in a first base and (b) a second component containing at least a second base, and wherein, upon mixture of the first component and the second component, the at least one tetracycline is rendered suitable for topical administration.

The method of this embodiment contemplates the administration of the first and second components either simultaneously or in either order. In other words, the first and second components may be applied to the accessible body surface of the human or animal as a mixture together or they may separately be applied to the accessible body surface of the human or animal, in either order. What is more, when applied together, the first and second components may be mixed together at the time of application to the body surface or they may be mixed prior to the application to the body surface. When the first and second components are mixed prior to application, one of ordinary skill in the art will recognize that mixture should be accomplished just prior to application. As used herein, “topical administration” refers to administration onto any accessible body surface of any human or animal species, preferably the human species, for example, the skin or mucosal epithelia. In certain embodiments of this invention, “topical” refers to an external application to the skin epithelium.

As used herein, “dermatological condition” refers to cosmetic and pathological disorders of the skin. Dermatological conditions include topical inflammatory skin conditions such as eczema, contact dermatitis, rosacea, psoriasis and acne including acne rosacea. As used herein, “acne” is a disorder of the skin characterized by papules, pustules, cysts, nodules, comedones, and other blemishes or skin lesions. These blemishes and lesions are often accompanied by inflammation of the skin glands and pilosebaceous follicles, as well as, microbial, especially bacterial, infection. For the purposes of this specification, acne includes all known types of acne. Some types of acne include, for example, acne vulgaris, cystic acne, acne atrophica, bromide acne, chlorine acne, acne conglobata, acne cosmetica, acne detergicans, epidemic acne, acne estivalis, acne fulminans, halogen acne, acne indurata, iodide acne, acne keloid, acne mechanica, acne papulosa, pomade acne, premenstral acne, acne pustulosa, acne scorbutica, acne scrofulosorum, acne urticata, acne varioliformis, acne venenata, propionic acne, acne excoriee, gram negative acne, steroid acne, nodulocystic acne and acne rosacea. Acne rosacea is characterized by inflammatory lesions (erythema) and telangiectasia. Telangiectasia is abnormally and permanently dilated blood vessels associated with a number of diseases. For example, facial telangiectasia is associated with age, acne rosacea, sun exposure, and alcohol use. The present invention can also be used to treat certain other types of acneiform dermal disorders, e.g. perioral dermatitis, seborrheic dermatitis in the presence of acne, gram negative folliculitis, sebaceous gland dysfunction, hiddradenitis suppurativa, pseudo-folliculitis barbae, or folliculitis.

Specific embodiments of the invention will now be demonstrated by reference to the following general methods of manufacture and examples. It should be understood that these examples are disclosed solely by way of illustrating the invention and should not be taken in any way to limit the scope of the present invention.

### EXAMPLE 1

A first component was prepared using the ingredients set forth in Table 1 below.

TABLE 1

| Ingredient | % w/w |
|---|---|
| ST-Elastomer 10 | 75.00 |
| ST-Cyclomethicone - NF | to 100.00 |
| Isopropyl Myristate | 10.00 |
| Minocycline HCl* | 1.42 |

*1.20% w/w minocycline free base

First, the cyclomethicone and the minocycline HCl were mixed in a beaker, following which the isopropyl myristate was added, to form a mixture. Next, the mixture was added to the ST-Elastomer 10 with stirring at room temperature (about 20° C.). The stirring continued until the mixture was substantially mixed with the ST-Elastomer 10.

A second component was prepared using the ingredients set forth in Table 2 below.

TABLE 2

| Ingredient | % w/w |
|---|---|
| Carbopol 974P | 2.00 |
| Ethanol | 20.00 |
| Water, USP | to 100.00 |
| Sodium Lauryl Sulfate | 0.50 |
| NaOH (aq) 20% w/w | add to pH ~6.4 |

First, the ethanol, water and sodium lauryl sulfate were mixed in a beaker. The Carbopol 974P was added slowly, while stirring using a Heildolph mixer. Finally, the pH was adjusted from about pH 3.4 using the NaOH (aq) 20% w/w to a pH of about 6.4. Stirring was continued until gelation occurred.

A topical composition (pH 6.4) was prepared by mixing equal weights of the first component of Table 1 and the second component of Table 2 prepared above at room temperature (about 20° C.).

### EXAMPLE 2

A first component was prepared using the ingredients set forth in Table 1 from Example 1. A second component was prepared using the ingredients set forth in Table 3 below.

TABLE 3

| Ingredient | % w/w |
|---|---|
| Carbopol 974P | 1.00 |
| Ethanol | 20.00 |
| Water, USP | to 100.00 |
| Docusate sodium | 1.00 |
| Triethanolamine | add to pH ~6.4 |
| Methyl parabens | 0.18 |
| Propyl parabens | 0.02 |
| Butyl parabens | 0.02 |

First, the ethanol, water, docusate sodium and preservatives (methyl parabens, propyl parabens and butyl parabens) were mixed in a beaker. The Carbopol 974P was added slowly, while stirring using a Heildolph mixer. Finally, the pH was adjusted from about pH 3.4 using the triethanolamine to a pH of about 6.4. Stirring was continued until gelation occurred.

A topical composition (pH 6.4) was prepared by mixing equal weights of the first component of Table 1 and the second component of Table 3 prepared above at room temperature (about 20° C.).

### EXAMPLE 3

A first component was prepared using the ingredients set forth in Table 1 from Example 1. A second component was prepared using the ingredients set forth in Table 4 below.

TABLE 4

| Ingredient | % w/w |
|---|---|
| Hydroxyethyl cellulose | 2.00 |
| Ethanol | 20.00 |
| Water, USP | to 100.00 |
| Docusate sodium | 0.50 |
| NaOH | add to pH ~6.4 |

First, the ethanol, water and docusate sodium were mixed in a beaker. The hydroxyethyl cellulose was added slowly, while stirring using a Heildolph mixer. Finally, the pH was adjusted from about pH 3.4 using the NaOH to a pH of about 6.4. Stirring was continued until gelation occurred.

A topical composition (pH 6.4) was prepared by mixing equal weights of the first component of Table 1 and the second component of Table 4 prepared above at room temperature (about 20° C.).

### EXAMPLE 4

A first component was prepared using the ingredients set forth in Table 5 below.

TABLE 5

| Ingredient | % w/w |
|---|---|
| Doxycycline Hyclate* | 0.231 |
| ST-Elastomer 10 | 80.000 |
| ST-Cyclomethicone-5-NF | to 100.000 |
| Isopropyl Myristate | 1.000 |

*0.20% w/w doxycycline free base

First, the cyclomethicone and the doxycycline hyclate were mixed in a beaker, following which the isopropyl myristate was added, to form a mixture. Next, the mixture was added to the ST-Elastomer 10 with stirring at room temperature (about 20° C.). The stirring continued until the mixture was substantially mixed with the ST-Elastomer 10.

### EXAMPLE 5

A first component was prepared using the ingredients set forth in Table 6 below.

TABLE 6

| Ingredient | % w/w |
|---|---|
| Doxycycline Monohydrate* | 0.208 |
| ST-Elastomer 10 | 80.000 |
| ST-Cyclomethicone-5-NF | to 100.000 |
| Isopropyl Myristate | 1.000 |

*0.20% w/w doxycycline free base

First, the cyclomethicone and the doxycycline monohydrate were mixed in a beaker, following which the isopropyl myristate was added, to form a mixture. Next, the mixture was added to the ST-Elastomer 10 with stirring at room temperature (about 20° C.). The stirring continued until the mixture was substantially mixed with the ST-Elastomer 10.

### EXAMPLE 6

A first component was prepared using the ingredients set forth in Table 7 below.

TABLE 7

| Ingredient | % w/w |
|---|---|
| Minocycline HCl* | 0.237 |
| Cyclomethicone | to 100.000 |
| ST-Elastomer 10 | 75.000 |
| Isopropyl myristate | 1.000 |

*0.20% w/w minocycline free base

First, the cyclomethicone and the minocycline HCl were mixed in a beaker, following which the isopropyl myristate was added to form a mixture. Next, the mixture was added to the ST-Elastomer 10 with stirring at room temperature (about 20° C.). The stirring continued until the mixture was substantially mixed with the ST-Elastomer 10.

### EXAMPLE 7

A first component was prepared using the ingredients set forth in Table 8 below.

TABLE 8

| Ingredient | % w/w |
|---|---|
| Minocycline HCl* | 0.473 |
| ST-Elastomer 10 | 80.00 |
| ST-Cyclomethicone-5-NF | to 100.00 |
| Isopropyl Myristate | 1.00 |

*0.40% w/w minocycline free base

First, the cyclomethicone and the minocycline HCl were mixed in a beaker, following which the isopropyl myristate was added, to form a mixture. Next, the mixture was added to the ST-Elastomer 10 with stirring at room temperature (about 20° C.). The stirring continued until the mixture was substantially mixed with the ST-Elastomer 10.

A second component was prepared using the ingredients set forth in Table 9 below.

TABLE 9

| Ingredient | % w/w |
|---|---|
| Carbopol 974P | 2.00 |
| Water, USP | to 100.00 |
| Ethanol | 20.00 |
| Emulsifier 10 | 1.00 |
| NaOH | add to pH ~6.0 |

First, the ethanol, water and Emulsifier 10 (Dow-Corning) were mixed in a beaker. The Carbopol 974P was added slowly, while stirring using a Heildolph mixer. Finally, the pH was adjusted from about pH 3.4 using the NaOH (aq) 20% w/w to a pH of about 6.0. Stirring was continued until gelation occurred.

A topical composition (pH 6.0) was prepared by mixing equal weights of the first component of Table 8 prepared above and the second component of Table 9 prepared above at room temperature (about 20° C.).

### EXAMPLE 8

A first component was prepared using the ingredients set forth in Table 10 below.

TABLE 10

| Ingredient | % w/w |
|---|---|
| Minocycline HCl* | 2.37 |
| ST-Elastomer 10 | 75.00 |
| ST-Cyclomethicone-5-NF | to 100.00 |
| Isopropyl Myristate | 1.00 |

*2.00% w/w minocycline free base

First, the cyclomethicone and the minocycline HCl were mixed in a beaker, following which the isopropyl myristate was added, to form a mixture. Next, the mixture was added to the ST-Elastomer 10 with stirring at room temperature (about 20° C.). The stirring continued until the mixture was substantially mixed with the ST-Elastomer 10.

A second component was prepared using the ingredients set forth in Table 9 in Example 7 above. A topical composition (pH 6.0) was prepared by mixing equal weights of the first component of Table 10 and the second component of Table 9 at room temperature (about 20° C.).

### EXAMPLE 9

A first component was prepared using the ingredients set forth in Table 11 below.

TABLE 11

| Ingredient | % w/w |
|---|---|
| Minocycline HCl* | 0.473 |
| ST-Elastomer 10 | 75.00 |
| ST-Cyclomethicone-5-NF | to 100.00 |

*0.40% w/w minocycline free base

First, the cyclomethicone and the minocycline HCl were mixed in a beaker to form a mixture. Next, the mixture was added to the ST-Elastomer 10 with stirring at room temperature (about 20° C.). The stirring continued until the mixture was substantially mixed with the ST-Elastomer 10.

A second component was prepared using the ingredients set forth in Table 12 below.

TABLE 12

| Ingredient | % w/w |
|---|---|
| Carbopol Ultrez 10 NF | 1.00 |
| Water, USP | to 100.00 |
| Ethanol | 20.00 |
| Pluronic F68 | 0.50 |
| Triethanolamine | add to pH ~6.4 |

First, the ethanol, water and Pluronic were mixed in a beaker. The Carbopol Ultrez was then added slowly, while stirring using a Heildolph mixer. Finally, the pH was adjusted using the triethanolamine to a pH of about 6.4.

A topical composition (pH 6.4) was prepared by mixing equal weights of the first and second components of Tables 11 and 12 above at room temperature (about 20° C.).

### EXAMPLE 10

A first component was prepared using the ingredients set forth in Table 13 below.

TABLE 13

| Ingredient | % w/w |
|---|---|
| Minocycline HCl* | 1.89 |
| ST-Elastomer 10 | 75.00 |
| Cyclomethicone-5 NF | to 100.00 |
| Isopropyl Myristate | 20.00 |

*1.60% w/w minocycline free base

First, the cyclomethicone and the minocycline HCl were mixed in a beaker, following which the isopropyl myristate was added, to form a mixture. Next, the mixture was added to the ST-Elastomer 10 with stirring at room temperature (about 20° C.). The stirring continued until the mixture was substantially mixed with the ST-Elastomer 10.

A second component was prepared using the ingredients set forth in Table 14 below.

TABLE 14

| Ingredient | % w/w |
|---|---|
| Carbopol 974P | 1.00 |
| Water, USP | to 100.00 |
| Ethanol | 20.00 |
| Pluronic F68 | 1.00 |
| Triethanolamine | add to pH ~6.4 |

First, the ethanol and water were mixed in a beaker. The Carbopol 974P was then added slowly while stirring using a Heildolph mixer. The Pluronic was then added and mixed for a further 2 minutes. Finally, the pH was adjusted using the triethanolamine to a pH of about 6.4.

A topical composition (pH 6.4) was prepared by mixing equal weights of the first and second components of Tables 13 and 14 at room temperature (about 20° C.).

### EXAMPLE 11

A first component was prepared using the ingredients set forth in Table 15 below.

TABLE 15

| Ingredient | % w/w |
|---|---|
| Minocycline HCl* | 1.89 |
| Cyclomethicone | to 100.000 |
| ST-Elastomer 10 | 75.000 |

*1.60% w/w minocycline free base

First, the cyclomethicone and the minocycline HCl were mixed in a beaker to form a mixture. Next, the mixture was added to the ST-Elastomer 10 with stirring at room temperature (about 20° C.). The stirring continued until the mixture was substantially mixed with the ST-Elastomer 10.

A second component was prepared using the ingredients set forth in Table 16 below.

TABLE 16

| Ingredient | % w/w |
|---|---|
| Water, USP | to 100.00 |
| Ethanol | 20.00 |
| Sodium Lauryl Sulfate | 1.00 |
| Carbopol 974P | 1.00 |
| Triethanolamine | add to pH ~6.4 |

First, the ethanol, water and sodium lauryl sulfate were mixed in a beaker. The Carbopol 974P was added slowly, while stirring using a Heildolph mixer. Finally, the pH was adjusted from about pH 3.4 using the triethanolamine to a pH of about 6.4.

A topical composition (pH 6.4) was prepared by mixing equal weights of the first component and the second component of Tables 15 and 16 at room temperature (about 20° C.).

### EXAMPLE 12

A first component was prepared using the ingredients set forth in Table 15 of Example 11 above. A second component was prepared using the ingredients set forth in Table 17 below.

TABLE 17

| Ingredient | % w/w |
|---|---|
| Water, USP | to 100.00 |
| Ethanol | 20.00 |
| Docusate sodium | 1.00 |
| Carbopol 974P | 1.00 |
| Triethanolamine | add to pH ~6.4 |

First, the ethanol, water and docusate sodium were mixed in a beaker. The Carbopol 974P was added slowly, while stirring using a Heildolph mixer. Finally, the pH was adjusted from about pH 3.4 using the triethanolamine to a pH of about 6.4. Stirring was continued until gelation occurred.

A topical composition (pH 6.4) was prepared by mixing equal weights of the first component of Table 15 and the second component of Table 17 at room temperature (about 20° C.).

### EXAMPLE 13

A first component is prepared using the ingredients set forth in Table 18 below.

TABLE 18

| Ingredient | % w/w |
|---|---|
| Caprylic/capric triglyceride | 24.50 |
| Mineral oil | 10.00 |
| Cyclomethicone | 32.00 |
| Beeswax | 1.50 |
| Sorbitan monooleate | 6.00 |
| Petrolatum | 25.00 |
| Minocycline hydrochloride | 1.00 |

All ingredients except the minocycline hydrochloride are weighed in a vessel and then heated to 70-75° C. with mixing until a uniform consistency is produced. The mixture is then cooled to 35° C. before gradually adding the minocycline hydrochloride. The minocycline-containing mixture (active mixture) is then cooled to ambient temperature and added to aerosol compartment 1 of a dual-compartment foam canister. The compartment is sealed and appropriate amount of propellant (5-25% w/w of the composition mass) is pressurized in an aluminum aerosol can with hydrofluorocarbon.

A second component is prepared using the ingredients set forth in Table 19 below.

TABLE 19

| Ingredient | % w/w |
|---|---|
| Dimethicone 350 | 25.00 |
| Water, USP | 72.00 |
| Stearyl Alcohol | 0.20 |
| Sucrose ester | 0.80 |
| Myrj 49 | 0.80 |
| Xanthan Gum | 0.20 |
| Methocel ELV15 | 0.40 |
| Antioxidant | 0.02 |
| Preservatives | 1.00 |

Aqueous phase: Xanthan Gum, Methocel ELV15, Sucrose ester and Myrj 49P are dissolved in water with agitation. The solution is warmed to 60° C., followed by addition of antioxidant and preservative(s). Hydrophobic Phase: Dimethicone 350 is heated to 60° C., and stearyl alcohol is added. The warm hydrophobic phase is gradually poured and agitated into the warm aqueous phase. After homogenization, the mixture is allowed to cool to ambient temperature. The mixture, at ambient temperature, is added to aerosol compartment 2 of a dual container foam canister, and the compartment is sealed. An appropriate amount of propellant (5-25% w/w of the composition mass) is added under pressure into compartment 2.

The first component of Table 18 and the separate second component of Table 19 are mixed together, either upon expulsion from the dual container foam canister or before or during application to the skin. Alternatively, the first component of Table 18 and the separate second component of Table 19 are mixed together upon expulsion from either the dual container foam canister or from two separate canisters after sequential application, in either order, to the skin.

### EXAMPLE 14

A first component was prepared using the ingredients set forth in Table 20 below.

TABLE 20

| Ingredient | % w/w |
|---|---|
| Minocycline HCl Micronized | 1.18 |
| ST-Elastomer 10 | 75.00 |
| ST-Cyclomethicone 5 NF | 13.82 |
| Isopropyl Myristate NF | 10.00 |

First, the cyclomethicone and the minocycline HCl were mixed in a beaker, following which the isopropyl myristate was added to form a mixture. Next, the mixture was added to the ST-Elastomer 10 with stirring. The stirring was continued until the mixture was substantially mixed with the ST-Elastomer 10.

A second component was prepared using the ingredients set forth in Table 21 below.

TABLE 21

| Ingredient | % w/w |
|---|---|
| Carbopol 974P NF | 0.70 |
| Pemulen TR-2 NF | 0.30 |
| Ethanol 96% Extra Pure USP | 20.83 |
| Purified Water | 75.60 |
| Docusate Sodium USP | 1.00 |
| Methylparaben NF | 0.08 |
| Propylparaben NF | 0.02 |

First, the ethanol 96% Extra Pure USP, docusate sodium USP, methylparaben NF and propylparaben NF were mixed in a beaker. Next, the carbopol 974P NF and pemulen TR-2 NF were slowly added to water in a second beaker, while stirring using a Heildolph mixer under high shear. The ethanol solution was then added to the carbopol solution with mixing. Finally, the pH was adjusted with trolamine NF to a pH of about 7.0. Stirring continued until gelation occurred.

### EXAMPLE 15

A first component was prepared using the ingredients set forth in Table 22 below.

TABLE 22

| Ingredient | % w/w |
|---|---|
| Minocycline HCl Micronized | 4.73 |
| ST-Elastomer 10 | 75.00 |
| ST-Cyclomethicone 5 NF | 10.27 |
| Isopropyl Myristate NF | 10.00 |

First, the cyclomethicone and the minocycline HCl were mixed in a beaker, following which the isopropyl myristate was added, to form a mixture. Next, the mixture was added to the ST-Elastomer 10 with stirring. The stirring was continued until the mixture was substantially mixed with the ST-Elastomer 10.

A second component was prepared using the ingredients set forth in Table 23 below.

TABLE 23

| Ingredient | % w/w |
|---|---|
| Carbopol 974P NF | 0.70 |
| Pemulen TR-2 NF | 0.30 |
| Ethanol 96% Extra Pure USP | 20.83 |
| Purified Water USP | 74.85 |
| Docusate Sodium USP | 1.00 |
| Propylparaben NF | 0.02 |
| Trolamine NF | 2.30 |

First, the ethanol 96% Extra Pure USP, docusate Sodium USP and propylparaben NF were mixed in a beaker. Next, the carbopol 974P NF and pemulen TR-2 NF were slowly added to water in a second beaker, while stirring using a Heildolph mixer under high shear. The ethanol solution was then added to the carbopol solution with mixing. Finally, the pH was adjusted with trolamine NF to a pH of about 7.9. Stirring continued until gelation occurred.

### COMPARATIVE EXAMPLE 1

A tetracycline formulation was prepared using the ingredients set forth in Table 24 below.

TABLE 24

| Ingredient | % w/w |
|---|---|
| Cyclomethicone | 40.81 |
| Isopropyl myristate | 41.40 |
| Polyethylene (MW 35000) | 17.20 |
| Minocycline HCl | 0.59 |

First, a portion of isopropyl myristate, a portion of cyclomethicone and minocycline HCl were added to a beaker to form a suspension. Next, the remaining quantity of isopropyl myristate and cyclomethicone were added to a second container and heated to 95° C. Once the temperature was attained, the polyethylene was added with stirring until a clear solution was produced. The solution formed was allowed to cool to 60° C. under stirring, and then the first suspension was added. The mixture was continuously stirred until an ointment was formed.

### Stability Testing

### Storage Stability of Doxycycline Hyclate Gel

The first component of Example 4 was tested for stability. Specifically, aliquots of the composition were stored in aluminum tubes for up to 3 months at 25° C. and 60% RH and for up to 4.5 months at 40° C. and 75% RH. The doxycycline content was assessed by HPLC after storage. To do this, first the doxycycline was extracted from the first component by taking a sample using a displacement pipette and pipetting directly into a tared volumetric flask. Absolute ethanol (30 ml) was then added, and the sample was then sonicated for 2-3 minutes. Deionized water was then used to accurately adjust to the required volume of 50 ml in the volumetric flask. Finally, the sample was filtered through a 0.45 μm filter prior to injection onto the HPLC column. The HPLC parameters used were as follows:

| | |
|---|---|
| Analytical Column: | Packing Type = Gemini RP18 |
| | Particle Size = 5 μm |
| | Column Length = 250 mm |
| | Internal Diameter = 4.6 mm |
| Mobile Phase (doxycycline): | 60 volumes KH₂PO₄ buffer (0.1M) |
| | 40 volumes methanol (HPLC) |
| | Adjust to pH 7.8 using 5N NaOH |
| | Add 0.5 g tetra-butyl ammonium |
| | hydrogen sulphate per litre |
| | after adjusting pH. |
| Flow rate: | 1.0 mL/min |
| Column Temperature: | 45° C. |
| Injection Volume: | 10 μL |
| Detection Wavelength: | 270 nm |

The results of the storage stability are shown in Tables 25 and 26 below.

TABLE 25
First Component of Example 4 stored at 25° C./60% RH.

| Active % Recovery | | % Area 4-Epimer | | % Area 6-Epimer | |
|---|---|---|---|---|---|
| 0 month | 3 months | 0 month | 3 months | 0 month | 3 months |
| 100.8 | 101.5 | ND | ND | 0.45 | 0.46 |

TABLE 26
First Component of Example 4 stored at 40° C./75% RH.

| | | | | |
|---|---|---|---|---|
| 0 month | 1 month | 2 months | 3 months | 4.5 months |

Active % Recovery*

| | | | | |
|---|---|---|---|---|
| 100.8 | 103.3 | 103.4 | 99.1 | 105.4 |

% Area 4-Epimer

| | | | | |
|---|---|---|---|---|
| ND | ND | ND | ND | ND |

% Area 6-Epimer

| | | | | |
|---|---|---|---|---|
| 0.45 | 0.42 | 0.43 | 0.42 | 0.47 |

ND means not detected
*numbers are mean value for 2 samples

As can be seen, after 3 months of storage at 25° C. and 60% relative humidity, the amount of doxycycline recovered remained unchanged. After 4.5 months of storage at 40° C. and 75% relative humidity, the amount of doxycycline recovered remained substantially unchanged. After 3 months, the amount of the 4-epimer (a degradation product, 4-epidoxycycline) is undetectable both at 40° C. and 75% RH and at 25° C. and 60% RH. The amount of the 6-epimer (a degradation product, 6-epidoxycycline) did not change over this time period both at 40° C. and 75% RH and at 25° C. and 60% RH.

### Storage Stability of Doxycycline Monohydrate Gel

The first component of Example 5 was tested for stability. Specifically, aliquots of the composition were stored in aluminum tubes for up to 4.5 months at 40° C. and 75% RH. The doxycycline content was assessed by HPLC after storage in a manner similar to that described above. The results of the storage stability are shown in Table 27 below.

TABLE 27
First Component of Example 5 stored at 40° C./75% RH.

| | | | | |
|---|---|---|---|---|
| 0 month | 1 month | 2 months | 3 months | 4.5 months |

Active % Recovery*

| | | | | |
|---|---|---|---|---|
| 104.9 | 98.2 | 107.5 | 102.1 | 103.3 |

% Area 4-Epimer

| | | | | |
|---|---|---|---|---|
| ND | ND | ND | ND | ND |

% Area 6-Epimer

| | | | | |
|---|---|---|---|---|
| 0.32 | 0.32 | 0.30 | 0.31 | 0.37 |

ND means not detected
*numbers are mean value for 2 samples

As can be seen, after 4.5 months of storage at 40° C. and 75% relative humidity, the amount of doxycycline recovered remained substantially unchanged. After 4.5 months, the amount of the 4-epimer (4-epidoxycycline) is undetectable at 40° C./75% RH. The amount of the 6-epimer (6-epidoxycycline) did not change over this time period at 40° C./75% RH.

### Storage Stability of Minocycline HCl Gel

The first component of Example 6 was tested for stability. Specifically, aliquots of the composition were stored in aluminum tubes for up to 6 months at 25° C. and 60% RH and for up to 6 months at 40° C. and 75% relative humidity. The minocycline content was assessed by HPLC after storage. To do this, first the minocycline was extracted from the first component by taking a sample using a displacement pipette and pipetting directly into a tared volumetric flask. Absolute ethanol (30 ml) was then added, and the sample was then sonicated for 2-3 minutes. Deionized water was then used to accurately adjust to the required volume of 50 ml in the volumetric flask. Finally, the sample was filtered through a 0.45 μm filter prior to injection onto the HPLC column. The limit of detection was 0.02 μg/ml (minocycline HCl). The mobile phase for the HPLC method consisted of methanol and acetonitrile.

The HPLC parameters used were as follows:

| | |
|---|---|
| Analytical Column: | Packing Type = Gemini RP18 |
| | Particle Size = 5 μm |
| | Column Length = 250 mm |
| | Internal Diameter = 4.6 mm |
| Flow rate: | 1.0 mL/min |
| Column Temperature: | 45° C. |
| Injection Volume: | 10 μL |
| Detection Wavelength: | 270 nm |

A minocycline primary standard was made up by accurately weighing about 55 mg of minocycline HCl reference material into a 100 mL volumetric flask; dissolving and making up to volume with water, stoppering and mixing well. From this primary standard, a secondary standard was made up by accurately transferring 5.0 mL of the minocycline primary standard into a 100 mL volumetric flask and making up to volume with water. When required, a minocycline tertiary standard was made up by accurately transferring 5.0 mL of the minocycline primary standard into a 250 mL volumetric flask and making up to volume with water. The amount of minocycline was calculated using the formula below:

$$Sample Conc / µg \text{/} ml = \frac{{S}_{w} \times P}{100} \times \frac{5}{100} \times \frac{5}{250} \times \frac{{A}_{sample}}{{A}_{std}} \times 1000$$

where:
S_{w}=Standard amount in mg
P=Decimal purity of standard as minocycline
Aₛₐₘₚₗₑ=Area of sample peak
A_{std}=Area of standard peak

The results of the storage stability are shown below in Tables 28 and 29.

TABLE 28
First Component of Example 6 stored at 25° C./60% RH.

| Active % Recovery | | | % Area 4-Epimer | | |
|---|---|---|---|---|---|
| 0 month | 3 months | 6 months | 0 month | 3 months | 6 months |
| 92.7 | 91.2 | 95.1 | 1.6 | 1.6 | 1.7 |

TABLE 29
First Component of Example 6 stored at 40° C./75% RH.

| | | | | |
|---|---|---|---|---|
| 0 month | 1 month | 2 months | 4.5 months | 6 months |

Active % Recovery

| | | | | |
|---|---|---|---|---|
| 86.2 | 89.4 | 87.0 | 98.2 | 89.2 |

% Area 4-Epimer

| | | | | |
|---|---|---|---|---|
| 1.6 | 1.4 | 1.7 | 1.6 | 1.7 |

The amount of the minocycline recovered after 6 months storage at 25° C./60% RH and 40° C./75% RH did not differ significantly from the starting level. The amount of the 4-epimer (4-epiminocycline) in the samples stored at 25° C./60% RH and 40° C./75% RH did not change over 6 months storage.

### Permeability Testing

Permeation through a Silicone Membrane—Concentration Effect

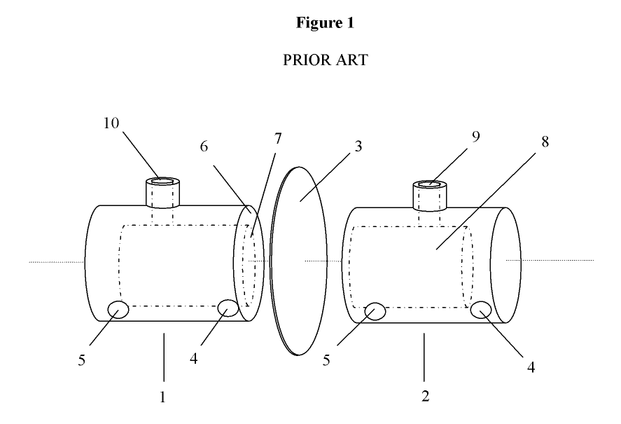

Permeation through a model skin (a silicone membrane) was assessed in the minocycline topical compositions of Examples 7 and 8. The first and second components of the respective topical compositions were mixed in equal parts by weight immediately prior to the permeation experiment. Permeation was conducted through a silescol membrane using a side-by-side diffusion cell (FIG. 1). After a period of 24 hours at 32° C., detectable levels of minocycline were found in the receiver medium. This was analyzed as minocycline using the same analytical method as set forth in the stability testing of minocycline gels above. The results (Table 30) suggest that the permeation rate is concentration dependent.

TABLE 30
Amount of Minocycline Detected in the Receiver Medium.

| | Average Amount of |
|---|---|
| | Minocycline (μg) detected |
| | at 24 hours |
| Topical composition of Example 7 | 7.4 |
| Topical composition of Example 8 | 24.6 |

### Permeation Through a Silicone Membrane—pH Effect

A further study investigated the permeation of minocycline from a liquid formulation comprising 0.47% (w/w) minocycline HCl dissolved in water through a silicone membrane as function of the pH. The diffusion of tetracycline from commercially available Topicycline® (0.2% w/w tetracycline HCl) across a silicone membrane was also compared under equivalent conditions. The permeation apparatus used (FIG. 1) was as follows. The apparatus consisted of two water-jacketed compartments 1 and 2, separated by a standard silicone membrane 3 (SMEM001 002, Esco Rubber, Barloworld Scientific Ltd, UK), which were clamped together. Each water-jacketed compartment 1 and 2 had respective water inlets 4 and water outlets 5 on the respective outer compartments 6, said outer compartments containing water. Inner compartment 7 for containing sample and inner compartment 8 for containing dissolution medium are also shown. The liquid formulation (or the Topicycline®) was immediately introduced into the inner compartment 7 via port 10. A pre-heated 0.9% saline solution (32° C.) was introduced via the sampling port 9 in the inner compartment 8. The temperature of the water jacket was kept at 32±1° C. by a temperature-controlled water bath. After the required time interval, the dissolution medium was sampled through the sampling port 9. The results from this study are shown in Table 31.

TABLE 31
Amount of Tetracycline or Minocycline
Detected in Receiver Medium

| | | Amount of Tetracycline or |
|---|---|---|
| | | Minocycline (μg) |
| Drug Product | pH | detected after 6 hours |
| Topicycline ® (Tetracycline HCl) | 2.5 | ND* |
| Minocycline HCl | 3.0 | ND* |
| Minocycline HCl | 5.0 | 0.17 |
| Minocycline HCl | 6.2 | 0.55 |
| Minocycline HCl | 7.0 | 0.31 |

*ND = not detected

This study indicated that the diffusion from a minocycline liquid formulation is pH sensitive, being maximal within the pH range of about 6.0 to about 7.0.

### Permeability Testing: Pig Skin

### Pig-Skin Permeation for the Topical Composition of Example 9

The topical composition of Example 9 (0.20% w/w minocycline) was tested for permeability across pig skin. The sample was separated from the receiver dissolution medium by full thickness (3 mm) pig skin.

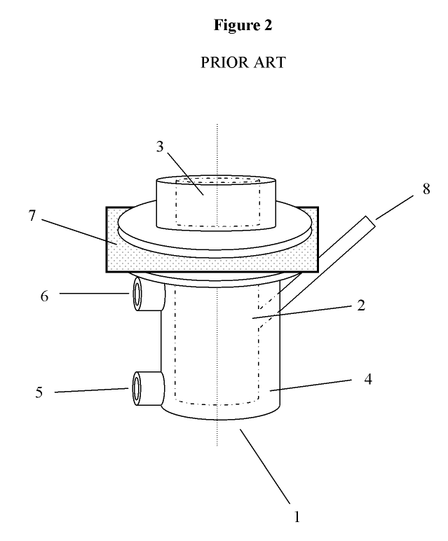

A conventional Franz diffusion cell, as shown in FIG. 2, was used for this test. The apparatus consisted of one water-jacketed compartment 1 containing the receiver medium in a lower inner compartment 2 and an upper inner compartment 3 for the test sample. The outer compartments 4 contained water for temperature control purposes and included both a water inlet 5 and a water outlet 6. The two compartments 2 and 3 were separated by full thickness pig skin 7 (3 mm), prepared by an appropriate method. The area available for permeation was 71 mm². A pre-heated (to 32° C.) phosphate-buffered saline (PBS) solution (PBS at pH 7.3±0.2 at 25° C.) was pipetted into the inner lower compartment 2 and the pig skin 7 placed on top ensuring that no air bubbles were present. After clamping together the upper 3 and lower 2 compartments, 1.50 g of the topical composition of Example 9 was syringed into the upper compartment 3. The temperature of the water jacket was kept at 32±1° C. by a temperature-controlled water bath. After the required time interval, the dissolution medium was sampled through the sampling port 8. Four replicates were performed, and the quantity of minocycline was determined in the dissolution medium by the analytical method described above after 4, 6 and 8 hours. The results are presented in Table 32.

TABLE 32
Amount of Minocycline Detected in Receiver Medium

| | Time | | |
|---|---|---|---|
| | 4 hrs | 6 hrs | 8 hrs |
| Average Amount of | 0.35 | 1.16 | 2.82 |
| Minocycline (n = 4) (μg) | | | |
| detected | | | |

This study indicates that the rate of permeation across pig skin is time-dependent.

### Permeability Testing: Mouse Skin

Mouse-Skin Permeation for the Topical Composition of Example 10 Each sample was separated from the receiver dissolution medium by full thickness mouse skin.

The apparatus consisted of one water-jacketed compartment containing the receiver medium 2 and an upper compartment 3 for the test sample as shown in FIG. 2. The two compartments were separated by mouse skin 7, prepared by an appropriate method. A pre-heated PBS solution (32° C.) was pipetted into the inner lower compartment 2 and the mouse skin 7 placed on top ensuring that no air bubbles were present. After clamping together the upper and lower compartments 3 and 2, 1.50 g of the test formulation was syringed into the upper compartment 3. The temperature of the water jacket was kept at 32±1° C. by a temperature-controlled water bath. After the required time interval, the dissolution medium was sampled through the sampling port 8. Three replicates were performed and the quantity of minocycline was assayed by the analytical method described above after 4 and 6 hours. The results for the topical compositions prepared according to Example 10 as compared with Topicycline® are presented in Table 33 below.

The topical composition of Example 10 (0.80% w/w minocycline) was tested for permeability across mouse skin.

TABLE 33
Average Amount (n = 3) of Tetracycline
or Minocycline Detected in Receiver Medium.

| | | Average Amount of |
|---|---|---|
| | | Tetracycline or |
| | | Minocycline (n = 3) (μg) |
| Topical Composition | Time | detected |
| Composition of Example 10 | 4 hrs | 0.42 |
| Composition of Example 10 | 6 hrs | 1.12 |
| Topicycline | 4 hrs | ND* |
| Topicycline | 6 hrs | 0.38 |

*ND means not detected

This study indicates that the rate of permeation across mouse skin is time-dependent.

### Effect of Anionic Excipients on the Permeation of Minocycline HCl Through Full Thickness Mouse Skin

The topical compositions of Examples 11 and 12 (0.8% w/w minocycline, as free base) were tested for permeation across mouse skin. The receiving medium was 5.0 ml of pH 7.3 phosphate-buffered saline heated to 32° C.

TABLE 34
Amount of Minocycline Detected in Receiver Medium from the
Topical Composition of Example 11 (Sodium Lauryl Sulfate).

| Sample | Average Amount of Minocycline (μg) |
|---|---|
| Time | Detected |
| 4 hrs | 0.67 |
| 6 hrs | 1.65 |
| 8 hrs | 3.11 |

TABLE 35
Amount of Minocycline Detected in Receiver Medium from the
Topical Composition of Example 12 (Docusate Sodium).

| Sample | Average Amount of Minocycline (μg) |
|---|---|
| Time | Detected |
| 4 hrs | 0.75 |
| 6 hrs | 2.20 |
| 8 hrs | 4.57 |

The results (Tables 34 and 35) established that adding an anionic surfactant such as docusate sodium to the topical composition of the present invention increases the flux of minocycline through full thickness mouse skin.

Commercially available Mecloderm® 1% crema (Shire, Italy) and Comparative Example 1 were tested for permeation across mouse skin.

TABLE 36
Amount of Minocycline Detected in Receiver
Medium from the Mecloderm.

| Sample | Average Amount of Minocycline (μg) |
|---|---|
| Time | Detected |
| 4 hrs | ND |
| 6 hrs | ND |
| 8 hrs | ND |

TABLE 37
Amount of Minocycline Detected in Receiver Medium from
the Topical Composition of Comparative Example 1

| Sample | Average Amount of Minocycline (μg) |
|---|---|
| Time | Detected |
| 4 hrs | ND |
| 6 hrs | ND |
| 8 hrs | ND |

The results (Tables 36 and 37) established that no flux of minocycline through full thickness mouse skin is evident for either Mecloderm or the gel formulation of Comparative Example 1, which falls within the scope of U.S. Pat. No. 5,122,519 (Ritter). In contrast, flux of minocycline through full thickness mouse skin has been demonstrated for the multi-part tetracycline formulations of the present invention.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims.

What is claimed is:

1. A multi-part tetracycline formulation comprising:
(a) a first component containing at least one tetracycline or a pharmaceutically acceptable salt or hydrate thereof substantially stabilized in a first base; and
(b) a second component containing at least a second base,
wherein, upon mixture of the first component and the second component, the at least one tetracycline is rendered suitable for topical administration.

2. The multi-part tetracycline formulation of claim 1, wherein topical administration is external administration to the skin.

3. The multi-part tetracycline formulation of claim 1, wherein more than 85% of the at least one tetracycline or its pharmaceutically acceptable salt or hydrate remains after storage at 25° C. and 60% relative humidity for 3 months.

4. The multi-part tetracycline formulation of claim 1, wherein the at least one tetracycline is substantially suspended in the first base of the first component.

5. The multi-part tetracycline formulation of claim 1, wherein the at least one tetracycline comprises a [4S-(4α,4aα,5aα,12aα)]-4-(dimethylamino)-1,4,4a,5,5a,6,11,12a-octahydro-3,10,12,12a-tetrahydroxy-1,11-dioxo-2-naphthacenecarboxamide having two different substituents at one or more of positions 4, 5, and 6.

6. The multi-part tetracycline formulation of claim 1, wherein the at least one tetracycline has the structural formula: 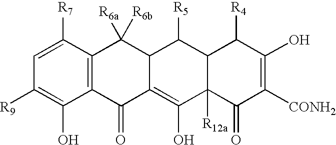
wherein R₄ is selected from the group consisting of a mono(lower alkyl)amino and a di(lower alkyl)amino;
R₉ is selected from the group consisting of hydrogen, a mono(lower alkyl)amino, a di(lower alkyl)amino and 2-(tert-butylamino)acetamido;
R₅ and R₁₂ₐ are independently selected from the group consisting of hydrogen and hydroxyl;
R₆ₐ and R_{6b} are independently selected from the group consisting of hydrogen, lower alkyl and hydroxyl, or can together form ═CH₂;
R₇ is selected from the group consisting of hydrogen, a halogen such as chloride, a mono(lower alkyl)amino and a di(lower alkyl)amino;
or a pharmaceutically acceptable salt or hydrate thereof.

7. The multi-part tetracycline formulation of claim 6, wherein the at least one tetracycline is selected from the group consisting of tetracycline; 7-methylamino-6-deoxy-6-demethyltetracycline; 7-ethylamino-6-deoxy-6-demethyltetracycline; 7-isopropylamino-6-deoxy-6-demethyltetracycline; 9-methylamino-6-deoxy-6-demethyltetracycline; 9-ethylamino-6-deoxy-6-demethyltetracycline; 9-isopropylamino-6-deoxy-6-demethyltetracycline; 7,9-di(ethylamino)-6-deoxy-6-demethyltetracycline; 7-dimethylamino-6-deoxy-6-demethyltetracycline; 9-dimethylamino-6-deoxy-6-demethyltetracycline; 7-methylamino-6-deoxytetracycline; 9-ethylamino-6-deoxytetracycline; 7,9-di(methylamino)-6-deoxytetracycline; 7-diethylamino-6-deoxytetracycline; 9-diethylamino-6-deoxytetracycline; 7,9-di(methylethylamino)-6-deoxytetracycline; 7-methylamino-9-ethylamino-6-deoxytetracycline; 9-methylamino-5-hydroxy-6-deoxytetracycline; 6-deoxy-5-hydroxytetracycline; oxytetracycline; 7-chlorotetracycline; 7-chloro-6-demethyltetracycline; 6-methyleneoxytetracycline; tigecycline and the pharmaceutically acceptable salts and hydrates.

8. The multi-part tetracycline formulation of claim 7, wherein the at least one tetracycline is minocycline or a pharmaceutically acceptable salt or hydrate thereof.

9. The multi-part tetracycline formulation of claim 1, wherein the at least one tetracycline is employed in an amount ranging from about 0.00001% to about 10% by weight of the multi-part tetracycline formulation.

10. The multi-part tetracycline formulation of claim 1, wherein the first base comprises at least one hydrophobic, non-hygroscopic liquid, at least one semi-solid hydrophobic, non-hygroscopic vehicle or a combination thereof.

11. The multi-part tetracycline formulation of claim 1, wherein the first component is substantially free of protic liquids.

12. The multi-part tetracycline formulation of claim 1, wherein the second base comprises at least one protic liquid, at least one non-protic liquid, at least one aqueous liquid, at least one non-aqueous liquid, at least one semi-solid vehicle or a combination thereof.

13. The multi-part tetracycline formulation of claim 1, further comprising at least one optional ingredient selected from the group consisting of mucoadhesive agents, surfactants, penetration enhancers, antioxidants, chelating agents, additional pharmaceutically active agents, pharmaceutically acceptable excipients and preservatives.

14. The multi-part tetracycline formulation of claim 13, wherein the at least one optional ingredient is included in the first component, in the second component or in both the first and the second components.

15. The multi-part tetracycline formulation of claim 13, wherein the additional pharmaceutically active agent is selected from the group consisting of anti-inflammatory compounds, antimicrobials, benzoyl peroxide, azelic acid, retinoids, immunomodulators, and calcineurin antagonists.

16. The multi-part tetracycline formulation of claim 1, wherein, upon mixture of the first component and the second component, the at least one tetracycline is substantially solubilized.

17. A method of making a multi-part tetracycline formulation comprising the steps of:
(a) preparing a first component containing at least one tetracycline or a pharmaceutically acceptable salt or hydrate thereof substantially stabilized in a first base; and
(b) preparing a second component containing at least a second base.

18. The method of making a multi-part tetracycline formulation of claim 17 further comprising the step of:
(c) mixing the first component and the second component to render the at least one tetracycline suitable for topical administration.

19. The method of making a multi-part tetracycline formulation of claim 18, wherein step (c) substantially solubilizes the at least one tetracycline.

20. The method of making a multi-part tetracycline formulation of claim 18, wherein the first component and the second component are mixed in equal parts.

21. A multi-part tetracycline formulation made according to the method of claim 17.

22. A multi-part tetracycline formulation made according to the method of claim 18.

23. A method of treating a dermatological condition comprising the step of:
administering a multi-part tetracycline formulation to an accessible body surface of a human or an animal in need of such treatment,
wherein the multi-part tetracycline formulation comprises (a) a first component containing at least one tetracycline or a pharmaceutically acceptable salt or hydrate thereof substantially stabilized in a first base and (b) a second component containing at least a second base, and
wherein, upon mixture of the first component and the second component, the at least one tetracycline is rendered suitable for topical administration.

24. The method of claim 23, wherein the first component and the second component are administered simultaneously.

25. The method of claim 23, wherein the first component and the second component are administered sequentially in either order.

26. The method of claim 23, wherein the first component and the second component are mixed together before administering the formulation to the accessible body surface.
